(19) United States
(12) Patent Application Publication
(10) Pub. No.: US 2016/0376612 A1
(43) Pub. Date: Dec. 29, 2016

(54) METHOD FOR PREPARING ETHANOL USING YEAST

(71) Applicant: Heui Kyu RYU, Jeollabuk-do (KR)

(72) Inventor: Heui Kyu RYU, Jeollabuk-do (KR)

(21) Appl. No.: 14/895,190

(22) PCT Filed: Jan. 27, 2015
(86) PCT No.: PCT/KR2015/000846

§ 371 Date: Dec. 1, 2015

(30) Foreign Application Priority Data
Mar. 17, 2014 (KR) 10-2014-0031032

(51) Int. Cl. C12P 7/14

(52) U.S. Cl. CPC C12P 7/14

(57) ABSTRACT

A method for producing ethanol using yeast is provided. The method includes a boiling process of heating a fermentation raw material, a saccharification process of saccharifying the boiled fermentation raw material with a coenzyme added thereto, and a fermentation process of inputting a mixed strain culture fluid into the saccharified fermentation raw material and fermentating the resultant product, wherein the mixed strain culture fluid is a culture fluid acquired by cultivating two or more kinds of Saccharomyces cerevisiae strains. According to the ethanol producing method, the bacterial growth is inhibited, thereby demonstrating excellent effects in improving the growth, cultivation and fermentation yields of the yeast. In addition, according to the ethanol producing method, a high yield of pure alcohol is demonstrated, thereby effectively saving the distillation cost for eliminating impurities.

### CROSS-REFERENCE TO RELATED APPLICATION

This application claims priority to and the benefit of Korean Patent Application No. 10-2014-0031032 filed on Mar. 17, 2014 in the Korean Intellectual Property Office, and all the benefits accruing therefrom under 35 U.S.C. 119, the contents of which in its entirety are herein incorporated by reference.

### BACKGROUND

1. Field

The present invention relates to a method for producing ethanol with an improved yield by inputting a culture fluid acquired by simultaneously cultivating two or more kinds of Saccharomyces cerevisiae strains in a fermentation process.

2. Description of the Related Art

Ethanol fermentation has traditionally placed emphasis on finding out optimal fermentation conditions by using a single type of yeast having such a property as glucose tolerance or temperature tolerance, or using the yeast that is effective in improving the yield. In recent years, however, efforts for solving problems posed by bacteria (contaminants) present in fermentation raw materials are being extensively attempted.

Specifically, the bacteria delay the growth of yeast, which may lower ethanol yields, resulting in a considerable loss of raw materials. Thus, additional costs may be incurred in the final stage of waste water treatment due to financial losses and sugar not involving in a subsequent fermentation process. More specifically, in order to eliminate the bacteria, disinfection and purge using high-temperature steam may be performed during fermentation and a fermentation tank, a yeast progression tank, a saccharification tank, a boiling vessel and transport pipes need to be kept in a clean state. However, since the fermentation raw material, such as unpolished rice or polished rice, is exposed to external air during storage, there is an increasing probability of cross contamination. In addition, typically used starch fermentation raw materials including fruits or plants of the ground, such as tapioca or sweet potato, are readily to decay due to an increase in the moisture or are prone to contamination. In addition, since the fermentation raw materials cannot washed with water for storage and are stained with dirt, they may be contaminated at a stage of supplying the same.

Further, since unpolished rice or polished rice is usually stored for a prolonged period of time for reasons of supply and demand circumstances or is left outdoor for a prolonged period of time in all seasons, a large quantity of microbes may be distributed and propagated outside the grain. The microbes are similar to yeast in view of growth temperature and pH and are both anaerobic and aerobic, they are exposed to well breeding circumstances. In addition, since the microbes include protein-degradable bacteria, they may serve as a main factor for degenerating liquor flavor.

Korean Patent No. 1988-0005273 discloses a process for improving productivity by inhibiting growth of bacteria and contaminants by directly inputting an antibiotic. However, before the antibiotic is input before fermentation is started, the antibiotic may be abused. Since carbon dioxide generated during an intermediate production process may make a fermentation tank maintained with a high pressure of about 1 to 2 kgf/cm² (0.1 to 0.2 Mpa) until a fermentation period is terminated, it is quite difficult to input the antibiotic under a high-pressure condition even when contamination is confirmed during the intermediate production process. However, very few methods for treating contaminants have to date been presented.

### SUMMARY

Embodiments of the present invention provide a method for producing ethanol, which is effective in improving the growth, cultivation and fermentation yields of yeast by inhibiting the bacterial growth.

The above and other aspects of the present invention will be described in or be apparent from the following description of exemplary embodiments.

According to an aspect of the present invention, there is provided a method for producing ethanol using yeast, the method including a boiling process of heating a fermentation raw material; a saccharification process of saccharifying the boiled fermentation raw material with a coenzyme added thereto; and a fermentation process of inputting a mixed strain culture fluid into the saccharified fermentation raw material and fermentating the resultant product, wherein the mixed strain culture fluid is a culture fluid acquired by cultivating two or more kinds of Saccharomyces cerevisiae strains.

The fermentation raw material may include starch.

The boiling process may be performed by heating the fermentation raw material at a temperature in a range of 80° C. to 110° C. for 1 to 5 hours.

The coenzyme may be glucoamylase, α-amylase or a mixture thereof.

The fermentation process may be performed at a temperature in a range of 30° C. to 40° C. for 10 hours to 5 days.

As described above, the method for producing ethanol according to the present invention can inhibit the bacterial growth by inputting a culture fluid acquired by simultaneously cultivating two or more kinds of Saccharomyces cerevisiae strains in a fermentation process, thereby demonstrating excellent effects in improving the growth, cultivation and fermentation yields of yeast.

In addition, the method for producing ethanol according to the present invention demonstrates a relatively high yield of pure alcohol, thereby effectively saving the distillation cost for eliminating impurities.

### BRIEF DESCRIPTION OF THE DRAWINGS

The above and other features of the present invention will become more apparent by describing in detail exemplary embodiments thereof with reference to the attached drawings in which:

FIG. 1 is a scanning electron microscopy (SEM) photograph showing a change in ethanol produced by Example over the fermentation time elapsed;

FIG. 2 is a scanning electron microscopy (SEM) photograph showing a change in ethanol produced by Comparative Example 1 over the fermentation time elapsed;

FIGS. 3A and 3B show yields of pure alcohol (ethanol) produced by Example, as measured using gas chromatography;

FIGS. 4A and 4B show yields of pure alcohol (ethanol) produced by Comparative Example 1, as measured using gas chromatography; and

FIGS. 5A and 5B show yields of pure alcohol (ethanol) produced by Comparative Example 2, as measured using gas chromatography.

### DETAILED DESCRIPTION

Hereinafter, a preferred embodiment of the present invention and physical properties of various components will be described in detail such that it can easily be made and used by those skilled in the art. However, the present invention is not to be limited in sprit and scope by the specific embodiment described herein.

The method for producing ethanol using yeast includes a boiling process of heating a fermentation raw material; a saccharification process of saccharifying the boiled fermentation raw material with a coenzyme input thereto; and a fermentation process of inputting a mixed strain culture fluid into the saccharified fermentation raw material and fermentating the resultant product, wherein the mixed strain culture fluid is a culture fluid acquired by cultivating two or more kinds of Saccharomyces cerevisiae strains.

The boiling process is preferably performed by heating the fermentation raw material at a temperature in a range of 80° C. to 110° C. for 1 to 5 hours. If the boiling process is performed at a temperature below 80° C., it is not possible to maintain an appropriate temperature for activating an enzyme and the effect of disinfecting contaminants may be negligible. If the boiling process is performed at a temperature above 110° C., the fermentation raw material, i.e., sugar, may be carbonized, which causes the energy source of yeast to be lost, resulting in a reduction in the yield.

More specifically, the fermentation raw material may include starch, and the starch is preferably gelatinized by heating the starch at the aforementioned temperature. The fermentation raw material including starch may include, for example, polished rice, unpolished rice and tapioca, but not limited thereto.

The saccharification process is to saccharify the boiled fermentation raw material. The boiled fermentation raw material is preferably transformed into a saccharide using a liquid purified enzyme or a coenzyme. Namely, glucoamylase, α-amylase and a mixture thereof are preferred.

The coenzyme is preferably added in a content of 0.01 to 10% by weight based on a total weight of the fermentation raw material. If the content of the coenzyme is less than 0.01% by weight, the saccharification process may not be performed well. If the content of the coenzyme is more than 0.01% by weight, the coenzyme, which is expensive, needs to be added in an increased quantity, resulting in an increase in the production cost.

The fermentation process is preferably performed by inputting a mixed strain culture fluid into the saccharified fermentation raw material for fermentation at a temperature in a range of 30° C. to 40° C. for 10 hours to 5 days. If the fermentation is out of the range described above, the fermentation based on the strain is not properly performed. More preferably, an initial temperature of the fermentation process is maintained at 34° C. to 36° C. for 10 to 60 hours, which may effectively improve alcohol yield using the mixed strain culture fluid. In addition, since the fermentation process is performed at a higher temperature than the conventional fermentation process, the process cost for cooling can be effectively reduced.

More specifically, the mixed strain culture fluid is preferably a culture fluid acquired by simultaneously cultivating two or more kinds of Saccharomyces cerevisiae strains. The culture fluid including an antibiotic, which is prepared by simultaneously cultivating two or more kinds of Saccharomyces cerevisiae strains can effectively function in inhibiting bacterial growth.

In detail, if the two or more kinds of Saccharomyces cerevisiae strains are simultaneously cultivated and grown, they primarily compete with each other for their own growth. During such a procedure, a large quantity of antibiotics are produced.

Since the produced antibiotics have antibacterial properties, the growth of bacteria is inhibited, thereby improving fermentation yields using the strains. In addition, the antibiotics are derived from microorganisms, they are not considered as being pathogenic but can be used as preservatives of food.

Examples of the bacteria may include molds and mycotoxins toxins; Lactics; coccus and bacillus of genus lactic acid bacillus, i.e., Lactobacteria (genus Streptococcus, genus Leuconostoc, genus Pediococcus, etc.); and the acetic acid producing bacteria (Acetics), the Acetice includes Acetobacter, Acetomonas, etc. that produce acetic acid. Examples of the molds and the mycotoxins may include aspergilus typically found in grains and beans. These molds generates aflatoxin producing an organic acid and not being eliminated even at high temperatures. Lactic acid bacillus includes Lactobacteria (streptococcus, Leuconostoc, pediococcus), and hinders yeast fermentation. The coccus and bacillus of genus lactic acid bacillus may hinder yeast fermentation, as proteolytic bacteria, they may strengthen the proteolytic capacity by extracellularly secreting a proteinase, and may serve as a major factor for changing the taste of liquor. The acetic acid producing bacteria (Acetics) includes Acetobacter or Acetomonas, and they oxidaze ethanol, which is a product of fermentation, into acetic acid.

However, since the present invention is to use the culture fluid including an antibiotic, which is prepared by simultaneously cultivating two or more kinds of Saccharomyces cerevisiae strains, the present invention can effectively function in inhibiting bacterial growth and then reduce any harmful effect induced by such bacteria as above.

In addition, the fermentation process employs a culture fluid prepared by simultaneously cultivating two or more kinds of Saccharomyces cerevisiae strains, thereby improving the yield of alcohol and enhancing economic efficiency of a distillation process for recovering the alcohol. In detail, during the alcohol fermentation process, a variety of kinds of fusel-oils are produced, which may increase the distillation cost. In particular, isopropyl alcohol having a boiling point similar to that of pure alcohol (ethanol) may also be produced. However, the isopropyl alcohol may deteriorate the taste and flavor of the finally produced alcohol. Accordingly, a separate distillation process for eliminating the isopropyl alcohol needs to be performed, which may result in an increase in the production cost.

### MODES FOR EMBODYING THE INVENTION

Hereinafter, the method for producing ethanol according to the present invention will be described with reference to Example.

### EXAMPLE

Cultivation of Mixed strains

Each of 0.1 to 0.5g of two kinds of Saccharomyces cerevisiae strains (SD1055 and SD1056) was inoculated in a YPD liquid medium at a temperature of 34° C., and cultivated for 12 hours, acquiring culture fluids. Each of 10 ml of the acquired culture fluids was input together to a conical flask, resulted in 20 ml in total, and 300 ml of liquid medium was input thereto, followed by recultivating the resultant product at a temperature of 34° C. for 12 hours, acquiring a mixed strain culture fluid. Here, SD1055 and SD1056 deposited in Korea Research Institute of Bioscience & Biotechnology (KRIBB) were used as the Saccharomyces cerevisiae strains, and 1 L YPD medium contained 1% yeast extract, 1% polypeptone, and 2% glucose while adjusting the pH to 4.8.

### Production of Alcohol

0.04 ml of α-amylase having a titer of 12000SP(120 KNU/g) or higher was input to 100 g of a fermentation raw material, heated at a temperature of 80° C. to 110° C. for 4 hours, and been cooled. Then, 0.7 g of a Diastatic enzyme including a glucoamylase component having a titer of 4000SP(40 KNU/g) or higher and containing less than 10% moisture was input to the resultant product, subjected to a saccharification process at a temperature of 60° C. for one hour, and each 10 ml of the mixed strain culture fluid was input thereto, maintained at a temperature of 36° C. for 40 hours, followed by fermenting at a temperature of 34° C., thereby producing the alcohol. Here, tapioca imported from Vietnam and having 12.9% of moisture was used as the fermentation raw material.

### Comparative Example 1

Alcohol was produced in substantially the same manner as in Example using the culture fluid of a strain cultivated in the following manner.

### Cultivation of Strain

Each of 0.1 to 0.5 g of only SD1055 as a Saccharomyces cerevisiae strain was inoculated in a YPD liquid medium at a temperature of 34° C., and cultivated for 12 hours, acquiring a culture fluid. Then, 20 ml of the acquired culture fluid was input to a conical flask, and 300 ml of liquid medium was input thereto, followed by recultivating the resultant product at a temperature of 34° C. for 12 hours, acquiring the culture fluid of the strain SD1055.

### Comparative Example 2

Alcohol was produced in substantially the same manner as in Example using the culture fluid of a strain cultivated in the following manner.

### Cultivation of Strain

Each of 0.1 to 0.5 g of only SD1056 as a Saccharomyces cerevisiae strain was inoculated in a YPD liquid medium at a temperature of 34° C., and cultivated for 12 hours, acquiring a culture fluid. Then, 20 ml of the acquired culture fluid was input to a conical flask, and 300 ml of liquid medium was input thereto, followed by recultivating the resultant product at a temperature of 34° C. for 12 hours, acquiring the culture fluid of the strain SD1056.

### Test Example 1

Quantities of carbon dioxide generated when fermentation of the alcohols produced in Example and Comparative Examples 1 and 2, pure alcohol yields and quantities of isopropyl alcohol generated were measured and the measuring results are listed in Tables 1 and 2 below.

Here, theoretically the quantity of carbon dioxide produced during the yeast fementation is 54.28g per 100g of starch and the quantity of ethanol produced during the yeast fermentation is 56.83g/100g of starch. The other words, since the quantity of carbon dioxide generated represents a constant ratio of 0.955:1 with respect to the quantity of ethanol produced, the quantity of ethanol produced was calculated based on the quantity of carbon dioxide generated.

TABLE 1

| | Quantity of carbon dioxide (CO₂) generated (g) | | |
|---|---|---|---|
| Fermentation | | Comparative | Comparative |
| time elapsed | Example | Example 1 | Example 2 |
| 102 hrs | 38.830 | 35.570 | 38.210 |

TABLE 2

| | Fermentation | | Comparative | Comparative |
|---|---|---|---|---|
| | time elapsed | Example | Example 1 | Example 2 |
| Pure alcohol | 102 hrs | 99.06427 | 98.20333 | 99.01810 |
| yield (%) | | | | |
| Isopropyl alcohol | 102 hrs | 0.014860 | 0.024990 | 0.017690 |
| yield (%) | | | | |

### Test Example 2

Aflatoxin detecting tests were performed on the alcohols produced in Example and Comparative Examples 1 and 2 at a fermentation starting time (0 hr) and at an elapsed time of 95 hours after the fermentation of the alcohols started and the test results are listed in Table 3.

The aflatoxin detecting tests were committed to the Institute of Agricultural Science of Chungnam National University.

TABLE 3

| Quantity of aflatoxin | | Comparative | Comparative |
|---|---|---|---|
| detected (ppb) | Example | Example 1 | Example 2 |
| 0 hr | 7.11 | 7.11 | 7.11 |
| 95 hrs | Not detected | 0.160 | 0.610 |

As confirmed from Table 2, the alcohol produced in Example in which a mixed strain culture fluid was used demonstrated the yield of pure alcohol 0.04614% and 0.86094% than the alcohols produced in Comparative Examples 1 and 2, respectively. This suggests that about 1% or more of the annual average output, 100,000 D/M(20,000 kL) of the alcohol is increased. In addition, the quantity of energy used is decreased about 4% or greater. Further, the alcohol produced in Example may effectively reduce production costs, including, for example, the expense incurred with use of a pH adjusting agent. In addition, it is also confirmed that the cost requiring for a separate distillation process can be effectively saved even with a decrease in the isopropyl alcohol yield.

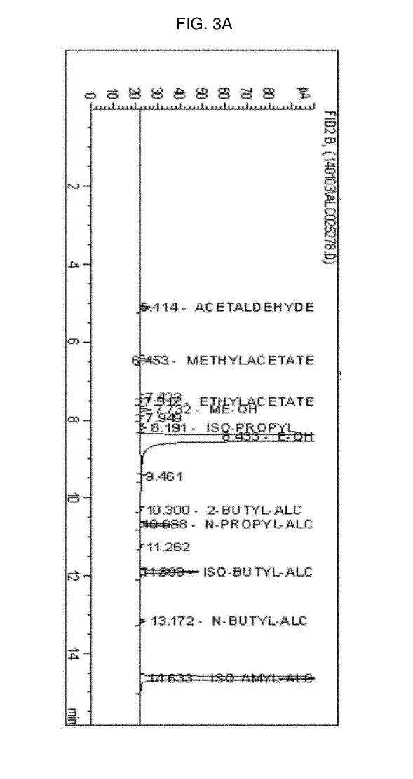

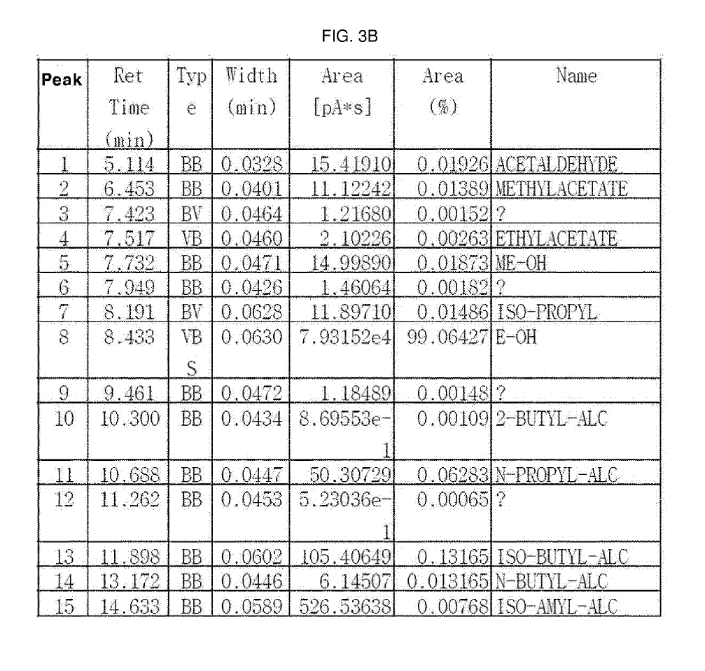

FIGS. 3A and 3B show yields of pure alcohol (ethanol) produced by Example, as measured using gas chromatography.

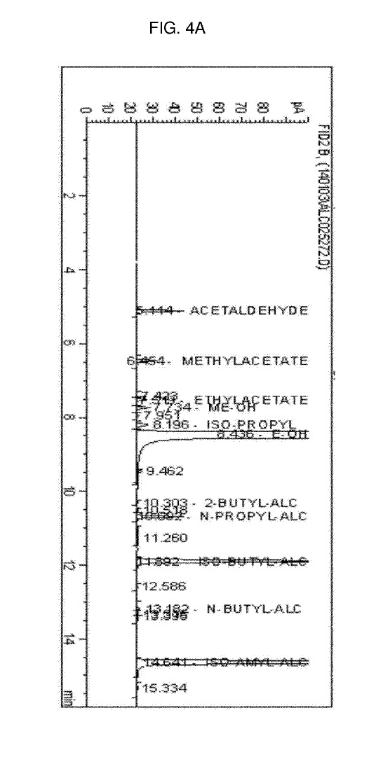

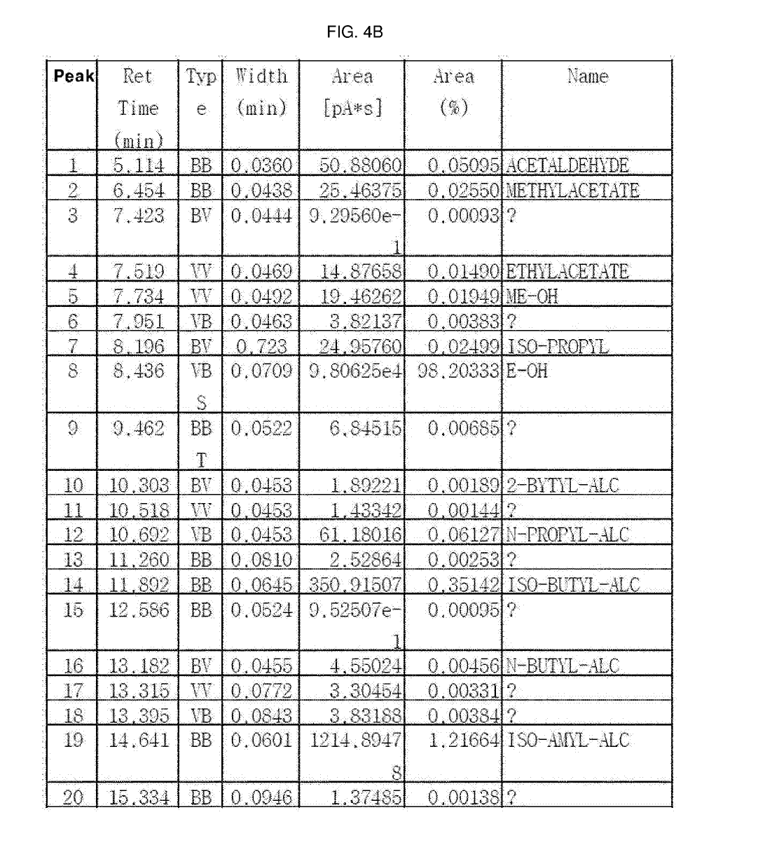

FIGS. 4A and 4B show yields of pure alcohol (ethanol) produced by

Comparative Example 1, as measured using gas chromatography.

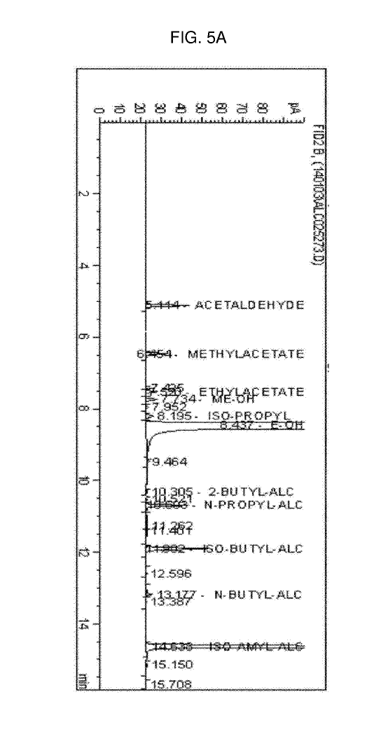

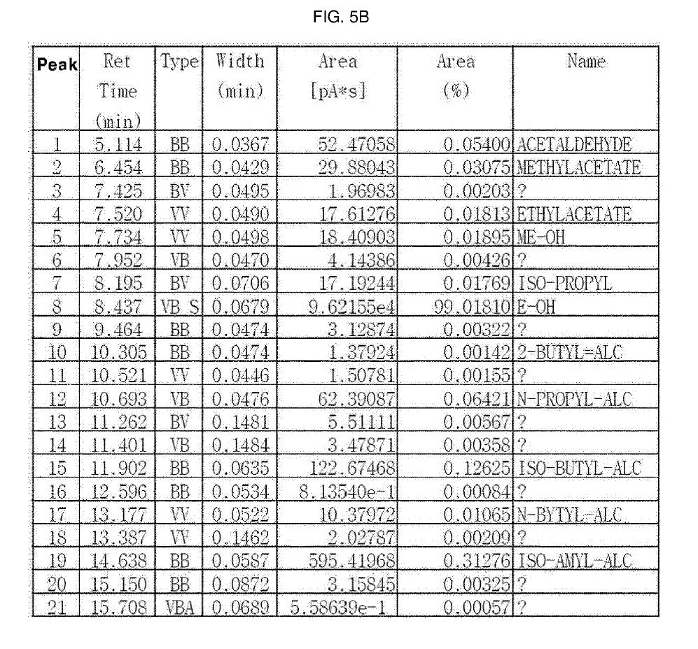

FIGS. 5A and 5B show yields of pure alcohol (ethanol) produced by Comparative Example 2, as measured using gas chromatography.

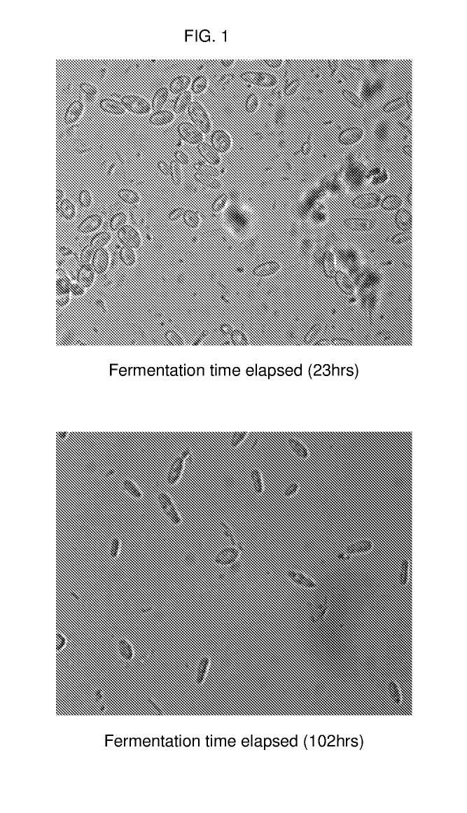

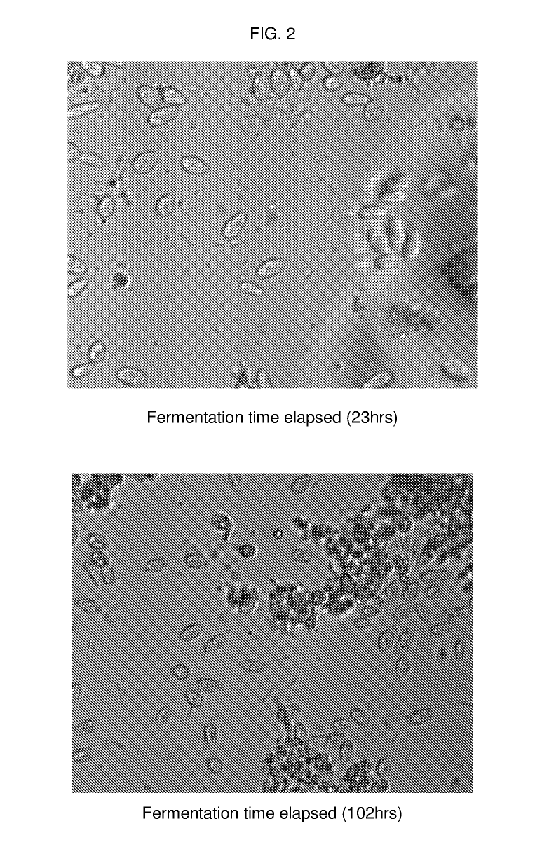

Referring to FIGS. 1 and 2, in Comparative Example 1 in which a single strain is used, the growth of bacteria was improved. However, in Example in which a mixed strain is used, it was visually confirmed that the growth of bacteria was inhibited through a fermentation process.

As confirmed from Table 3, since no aflatoxin was detected from the alcohol produced in Example when the fermentation time of 95 hours was elapsed after the fermentation was started, the alcohol produced in Example can effectively eliminate mold toxins.

Therefore, the method for producing ethanol according to the present invention can inhibit the bacterial growth by inputting a culture fluid acquired by simultaneously cultivating two or more kinds of Saccharomyces cerevisiae strains in a fermentation process, thereby demonstrating excellent effects in improving the growth, cultivation and fermentation yields of yeast. In addition, the method for producing ethanol according to the present invention demonstrates a high yield of pure alcohol, thereby effectively saving the distillation cost for eliminating impurities.

While the method for producing ethanol according to the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method for producing ethanol using yeast, the method comprising:
a boiling process of heating a fermentation raw material;
a saccharification process of saccharifying the boiled fermentation raw material with a coenzyme added thereto; and
a fermentation process of inputting a mixed strain culture fluid into the saccharified fermentation raw material and fermentating the resultant product,
wherein the mixed strain culture fluid is a culture fluid acquired by cultivating two or more kinds of Saccharomyces cerevisiae strains.

2. The method of claim 1, wherein the fermentation raw material includes starch.

3. The method of claim 1, wherein the boiling process is performed by heating the fermentation raw material at a temperature in a range of 80° C. to 110° C. for 1 to 5 hours.

4. The method of claim 1, wherein the coenzyme is glucoamylase, α-amylase or a mixture thereof.

5. The method of claim 1, wherein the fermentation process is performed at a temperature in a range of 30° C. to 40° C. for 10 hours to 5 days.
